(19) United States
(12) Patent Application Publication
(10) Pub. No.: US 2017/0231479 A1
(43) Pub. Date: Aug. 17, 2017

(54) ENDOSCOPE AND ENDOSCOPE SYSTEM INCLUDING ENDOSCOPE

(71) Applicant: OLYMPUS CORPORATION, TOKYO (JP)

(72) Inventor: Kazuki HONDA, Tokyo (JP)

(73) Assignee: OLYMPUS CORPORATION, TOKYO (JP)

(21) Appl. No.: 15/585,411

(22) Filed: May 3, 2017

Related U.S. Application Data
(63) Continuation of application No. PCT/JP2015/080071, filed on Oct. 26, 2015.

(30) Foreign Application Priority Data
Dec. 15, 2014 (JP) 2014-253277

(51) Int. Cl. A61B 1/06; G02B 23/24; H04N 5/225; A61B 1/00; A61B 1/04

(52) U.S. Cl. CPC A61B 1/06; A61B 1/00045; A61B 1/00027; A61B 1/04; H04N 5/2256; G02B 23/2469; G02B 23/2484; H04N 2005/2255; G02B 23/2423

(57) ABSTRACT

An endoscope includes: an insertion portion; a first observation portion configured to observe a first area including a front of the insertion portion; a second observation portion configured to observe a second area including a side of the insertion portion and be adjacent to the first area; a first illumination portion configured to irradiate the second area with first illumination light; and a second illumination portion configured to irradiate the second area with second illumination light, and the second illumination portion performs irradiation with the second illumination light of a light quantity equal to a light quantity for which the first illumination light in a range observed in the second observation portion and illumination light from an illumination portion configured to irradiate the first area with the illumination light are put together.

### CROSS REFERENCE TO RELATED APPLICATION

This application is a continuation application of PCT/JP2015/080071 filed on Oct. 26, 2015 and claims benefit of Japanese Application No. 2014-253277 filed in Japan on Dec. 15, 2014, the entire contents of which are incorporated herein by this reference.

### BACKGROUND OF THE INVENTION

1. Field of the Invention

The present invention relates to an endoscope capable of observing and picking up a plurality of images of different visual fields and an endoscope system including the endoscope.

2. Description of the Related Art

Conventionally, endoscopes configured including an insertion portion formed in an elongated shape are widely utilized in a medical field and an industrial field for example. Among them, an endoscope for medical use used in the medical field is configured to observe an organ inside a body cavity to be a subject by inserting the elongated insertion portion into the body cavity and to execute various kinds of treatments using a treatment instrument inserted into a treatment instrument insertion channel provided in the endoscope as needed. In addition, an endoscope for industrial use used in the industrial field is configured to observe and inspect a state inside an object, a flaw or corrosion for example, by inserting the elongated insertion portion inside the object, a jet engine or plant piping for example.

In addition, in recent years, various proposals regarding constitutional contrivances for making it easier to perform an inspection or the like conducted using a conventional endoscope have been disclosed. For example, an endoscope system including a so-called wide angle visual field endoscope configured to simultaneously acquire a front visual field image with a front in an insertion axis direction of an endoscope insertion portion as an observation visual field and a side visual field image with a side in a radial direction of the insertion portion that is a direction roughly orthogonal to the insertion axis direction of the endoscope insertion portion as the observation visual field is disclosed by Japanese Patent Application Laid-Open Publication No. 2013-544617 for example.

The endoscope and an endoscope system disclosed by Japanese Patent Application Laid-Open Publication No. 2013-544617 or the like are configured including a first camera that observes a front visual field, a front illumination portion that illuminates the front visual field, a second camera that observes a side visual field, and a side illumination portion that illuminates the side visual field. Then, by a plurality of light emitting diodes provided on a distal end portion of the endoscope insertion portion, the front and the side of the endoscope insertion portion are illuminated respectively. In this way, the above-described endoscope system is configured to irradiate a wide range at the front and the side of the distal end portion of the endoscope insertion portion with illumination light.

### SUMMARY OF THE INVENTION

An endoscope as one aspect of the present invention includes: an insertion portion configured to be inserted to an inside of a subject; a first observation portion provided on a front surface of the insertion portion and configured to observe a first area including a front which is a longitudinal axis direction of the insertion portion in an observation target; a second observation portion provided on a side surface of the insertion portion and configured to observe a second area including a side which is a direction crossing the longitudinal axis direction of the insertion portion in the observation target and be adjacent to the first area; a first illumination portion arranged on a distal end side with respect to the second observation portion on the side surface of the insertion portion and configured to irradiate the second area of the observation target with first illumination light; and a second illumination portion arranged on a proximal end side with respect to the second observation portion on the side surface of the insertion portion and configured to irradiate the second area of the observation target with second illumination light, and the second illumination portion performs irradiation with the second illumination light of a light quantity almost equal to a light quantity for which the first illumination light in a range observed in the second observation portion and illumination light from an illumination portion provided on the front surface of the insertion portion and configured to irradiate at least the first area with the illumination light are put together.

In addition, an endoscope system as one aspect of the present invention includes: the endoscope; an image signal generation portion configured to generate image signals for which a first image that is an image of the observation target in the first area and a second image that is an image of the observation target in the second area are lined up such that the first image and the second image are arranged at adjacent positions; and a display portion configured to display the image signals generated by the image signal generation portion.

### BRIEF DESCRIPTION OF THE DRAWINGS

FIG. 1 is a block configuration diagram schematically illustrating an entire configuration of an endoscope system of a first embodiment of the present invention;

FIG. 2 is a block configuration diagram schematically illustrating an internal configuration of a distal end portion of an insertion portion of an endoscope in the endoscope system in FIG. 1;

FIG. 3 is a diagram conceptually illustrating one example of brightness distribution on a display screen of a display portion in the endoscope system in FIG. 1;

FIG. 4 is a block configuration diagram schematically illustrating a part (an inside of the distal end portion) of a configuration of an endoscope of a second embodiment of the present invention;

FIG. 5 is a block configuration diagram schematically illustrating an entire configuration of an endoscope system of a third embodiment of the present invention; and

FIG. 6 is a block configuration diagram schematically illustrating an internal configuration of a distal end portion of an insertion portion of an endoscope in the endoscope system in FIG. 5.

### DETAILED DESCRIPTION OF THE PREFERRED EMBODIMENT(S)

Hereinafter, the present invention will be described with illustrated embodiments. Individual drawings used in following descriptions are schematically illustrated, and in order to indicate individual components recognizably on the drawings, dimensional relations and scales or the like of individual members are sometimes made different for each component and illustrated. Therefore, the present invention is not limited only to an illustrated form, regarding quantities of the components described on the individual drawings, shapes of the components, ratios of sizes of the components, relative positional relations of the individual components, or the like.

### First Embodiment

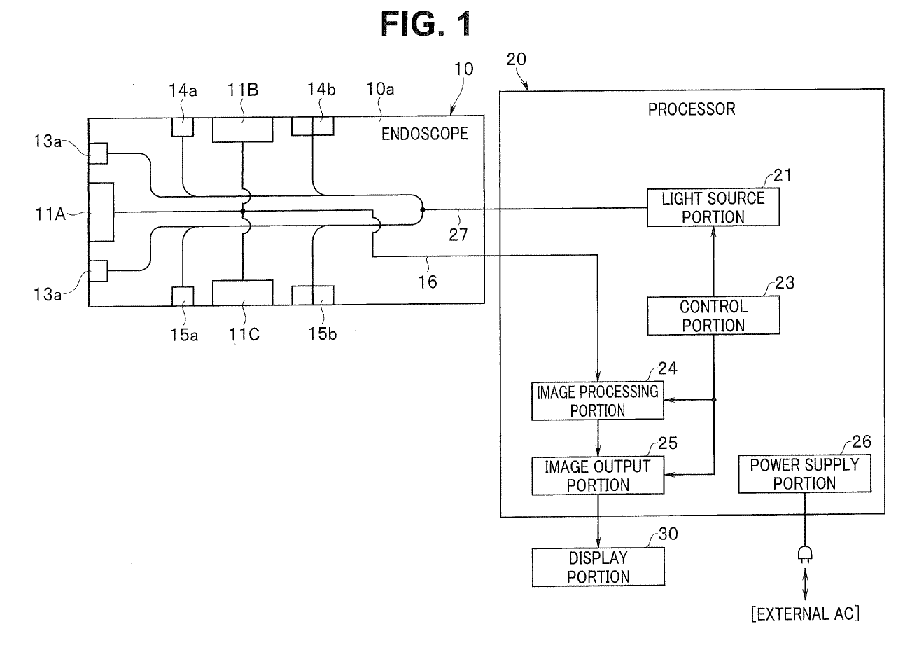

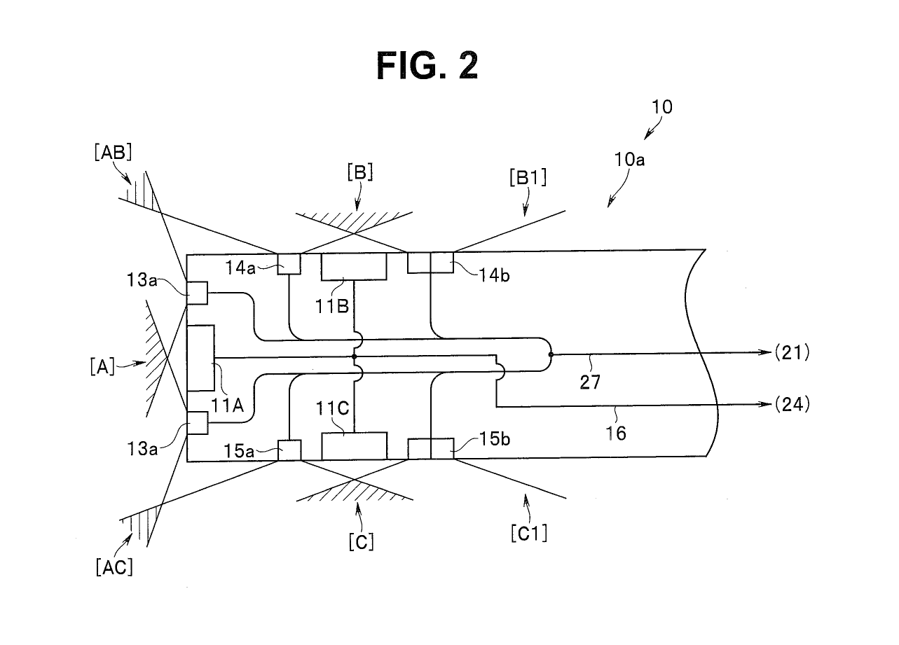

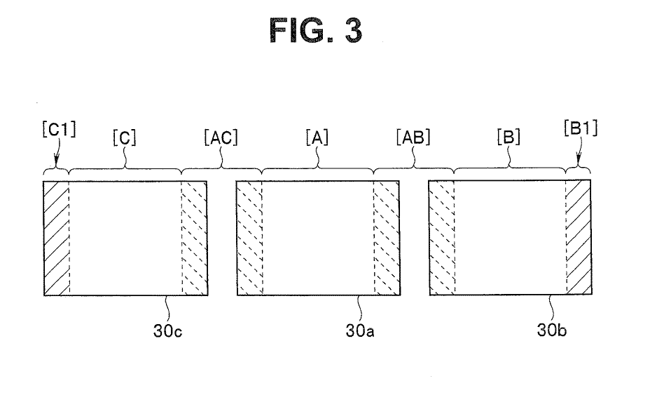

FIG. 1 is a block configuration diagram schematically illustrating an entire configuration of an endoscope system of a first embodiment of the present invention. FIG. 2 is a block configuration diagram schematically illustrating an internal configuration of a distal end portion of an insertion portion of an endoscope in the endoscope system of the present embodiment. In addition, FIG. 2 illustrates one example of an irradiation range of illumination light emitted from individual illumination portions together. FIG. 3 is a diagram conceptually illustrating one example of brightness distribution on a display screen of a display portion in the endoscope system of the present embodiment.

First, the configuration of the endoscope system of the present embodiment will be described below using FIG. 1 and FIG. 2. The present embodiment illustrates, for example, a so-called wide angle visual field endoscope configured to simultaneously acquire a front visual field image with a front in an insertion axis (longitudinal) direction of an endoscope insertion portion as an observation visual field and a side visual field image with a side in a radial direction of the insertion portion that is a direction crossing the insertion axis (longitudinal) direction of the endoscope insertion portion (for example, a roughly orthogonal direction or a direction forming an angle of several tens of degrees) as the observation visual field, and the endoscope system including the endoscope.

Note that the front visual field image refers to an image of an observation target (subject) in a first area including the front of the insertion portion roughly parallel to the longitudinal direction of the insertion portion, and the image is also called a first image. In addition, the side visual field image is an image of the observation target (subject) in a second area including the radial direction of the insertion portion that is the direction crossing the longitudinal axis direction of the insertion portion, and the image is also called a second image.

As illustrated in FIG. 1, an endoscope system 1 of the present embodiment is mainly configured by an endoscope 10 of the present embodiment, a processor 20, a display portion 30 or the like.

Note that the endoscope system 1 of the present embodiment is configured including, other than configuration units described above, a keyboard which is an external input device and a rack for mounting various kinds of the configuration units described above for example, however, since the configuration units are parts not directly related to the present invention, the illustration and the detailed descriptions are omitted.

For the endoscope 10, an endoscope of a conventional and general form configured by an insertion portion, an operation portion and a universal cable or the like is applied. That is, the insertion portion is formed of an elongated tubular configuration unit formed by connecting a distal end portion 10a, a bending portion, and a flexible tube portion in order from a distal end side. A proximal end of the insertion portion is connected to a distal end of the operation portion.

The insertion portion is a configuration portion configured to be inserted to an inside of a lumen (inside of a body cavity) of the subject when the endoscope 10 is used. The distal end portion 10a is disposed on a most distal end side of the insertion portion, is configured by a rigid member, and is a configuration unit with various kinds of configuration members arranged on an outer surface and in the inside.

Note that, in FIG. 1, the configuration of the endoscope 10 is simplified and illustrated, illustrations of the operation portion and the universal cable or the like are omitted, and an internal configuration of the distal end portion 10a is mainly illustrated.

The endoscope 10 in the endoscope system 1 of the present embodiment is configured to observe and pick up the plurality of images of the different visual fields which are the front visual field and the side visual field. Therefore, inside the distal end portion 10a of the insertion portion of the endoscope 10, image pickup portions configured including image pickup devices (11A, 11B, 11C) which are a plurality of photoelectric conversion elements and an image pickup optical system or the like (not shown in the figure), and a plurality of light sources (13a, 14a, 14b, 15a, 15b) which are controlled by a light source portion 21 provided outside the insertion portion to be described later and emit the illumination light to the outside or the like are disposed.

Here, an arrangement configuration of the plurality of image pickup devices (11A, 11B, 11C) and the plurality of light sources (13a, 14a, 14b, 15a, 15b) inside the distal end portion 10a of the present endoscope 10 will be simply described below mainly using FIG. 2.

The image pickup device indicated by a sign 11A among the plurality of image pickup devices (11A, 11B, 11C) is the image pickup device for acquiring the front visual field image which is a first observation portion and a front image acquisition portion, a light receiving surface of which is arranged toward the front, in order to acquire the front visual field image (first image) with the front (first direction) in the insertion axis direction of the endoscope insertion portion as the observation visual field, that is, a first image pickup portion configured to photoelectrically convert the first image.

In addition, the image pickup devices indicated by signs 11B and 11C among the plurality of image pickup devices (11A, 11B, 11C) are the image pickup devices for acquiring the side visual field image which are second observation portions and side image acquisition portions, the light receiving surfaces of which are arranged toward the side respectively, in order to acquire the side visual field image (second image) with the side (second direction different from the first direction) in the radial direction of the insertion portion that is the direction roughly orthogonal to the insertion axis direction of the endoscope insertion portion as the observation visual field, that is, a second image pickup portion configured to photoelectrically convert the second image and be different from the first image pickup portion.

Note that the image pickup device 11B and the image pickup device 11C are arranged oppositely back to back such that the individual light receiving surfaces oppose each other, and are configured to acquire the side visual field image respectively in the directions opposite to each other with the insertion axis direction as a center.

Each of the plurality of light sources (13a, 14a, 14b, 15a, 15b) is a luminous body such as a light emitting diode (LED), and is configured including at least one light emitting element.

Among the plurality of light sources (13a, 14a, 14b, 15a, 15b), the plurality of (two) light sources indicated by a sign 13a are the light sources for illuminating the front visual field, which irradiate a predetermined range of the front in the insertion axis direction of the endoscope insertion portion with illumination light. The plurality of (two) light sources 13a are arranged in a pair at both side parts of the image pickup device 11A for acquiring the front visual field image so as to hold the image pickup device 11A in between, within a plane virtually set to be roughly orthogonal to the insertion axis direction. By the configuration, the plurality of (two) light sources 13a mainly illuminate the observation target included in the predetermined range of the front visual field.

In addition, the plurality of (two) light sources indicated by signs 14a and 14b are the light sources for illuminating the side visual field, which irradiate a predetermined range of one side (a right side when a distal end side is viewed from a proximal end side regarding the insertion axis direction for example) in the radial direction of the insertion portion that is the direction roughly orthogonal to the insertion axis direction of the endoscope insertion portion with the illumination light. The plurality of (two) light sources 14a and 14b are arranged at both side parts of one image pickup device 11B for acquiring the side visual field image so as to hold the image pickup device 11B in between, within a plane virtually set to be parallel with the insertion axis direction. By such a configuration, the plurality of (two) light sources 14a and 14b mainly illuminate the observation target included in the predetermined range of the side visual field.

Then, the plurality of (two) light sources indicated by signs 15a and 15b are the light sources for illuminating the side visual field, which irradiate a predetermined range of the other side (a left side when the distal end side is viewed from the proximal end side regarding the insertion axis direction for example) in the radial direction of the insertion portion that is the direction roughly orthogonal to the insertion axis direction of the endoscope insertion portion with the illumination light. The plurality of (two) light sources 15a and 15b are arranged at both side parts of the other image pickup device 11C for acquiring the side visual field image so as to hold the image pickup device 11C in between, within a plane parallel with the insertion axis direction. By such a configuration, the plurality of (two) light sources 15a and 15b mainly illuminate the observation target included in the predetermined range of the side visual field.

Here, the light sources 14a and 15a arranged near the respective distal ends across the image pickup devices 11B and 11C among the plurality of light sources 14a, 14b, 15a and 15b for illuminating the side visual field are first illumination portions arranged between the image pickup device 11A (first observation portion) for acquiring the side visual field image and the image pickup devices 11B and 11C (second observation portion) for acquiring the front visual field image and configured to irradiate the observation target. The light sources 14a and 15a which are the first illumination portions are arranged at the sides of the endoscope insertion portion but are arranged in a pair across the first observation portion arranged at the front of the endoscope insertion portion. The first illumination portions are configured respectively by at least one first light emitting element which perform irradiation with first illumination light based on power supplied from a power supply portion 26 (electric power supply portion) to be described later. Here, the first light emitting element is a light emitting diode (LED) or the like.

In addition, the light sources 14b and 15b arranged near the respective proximal ends across the image pickup devices 11B and 11C, that is on opposite sides of the light sources 13a and the light sources 14a and 15a near the distal ends, among the plurality of light sources 14a, 14b, 15a and 15b for illuminating the side visual field are second illumination portions arranged at peripheral parts of the image pickup devices 11B and 11C (second observation portion) for acquiring the side visual field image and parts different from the light sources 14a and 15a (first illumination portions) and configured to perform irradiation with second illumination light of a light quantity different from a light quantity of the first illumination light emitted from the light sources 14a and 15a (first illumination portions). The second illumination portions are configured respectively by at least one second light emitting element which perform irradiation with the second illumination light based on the power supplied from the power supply portion 26 (electric power supply portion) to be described later. Here, the second light emitting element is a light emitting diode (LED) or the like.

Then, setting is performed so as to emit the different light quantities by the illumination light by the light sources 14a and 15a (first illumination portions) and the illumination light by the light sources 14b and 15b (second illumination portions). In this case, a light quantity difference between the light sources 14a and 15a near the distal end and the light sources 14b and 15b near the proximal end is realized by following configuration contrivances.

For example, a number of luminous bodies (first light emitting elements; LEDs for example) respectively installed as the light sources 14a and 15a (first illumination portions) near the distal end and a number of luminous bodies (second light emitting elements; LEDs for example) installed as the light sources 14b and 15b (second illumination portions) near the proximal end are changed. Specifically, while the light sources 14a and 15a near the distal end are configured by one luminous body (LED for example), and the light sources 14b and 15b near the proximal end are configured by two luminous bodies (LEDs for example) (a form illustrated in FIG. 2). Alternatively, the numbers of the respective luminous bodies of the light sources 14a and 15a near the distal end and the light sources 14b and 15b near the proximal end are turned to the same number, and the configuration is made such that control of changing a supply current to the light sources 14a and 15a near the distal end and a supply current to the light sources 14b and 15b near the proximal end can be performed. By the contrivances, the light quantities of the light sources 14a and 15a (first illumination portions) near the distal end and the light sources 14b and 15b (second illumination portions) near the proximal end can be adjusted.

In addition, as one example, setting is performed such that the second illumination light emitted from the light sources 14b and 15b (second illumination portions) can emit the light quantity larger than the light quantity of the first illumination light emitted from the light sources 14a and 15a (first illumination portions) near the distal end. For example, in contrast with the number of the luminous bodies (first light emitting elements; LEDs for example) respectively installed as the light sources 14a and 15a (first illumination portions) near the distal end, the larger number of the luminous bodies (second light emitting elements; LEDs for example) installed as the light sources 14b and 15b (second illumination portions) near the proximal end are installed. Specifically, when the light sources 14a and 15a near the distal end are configured by one luminous body (LED for example) for example, the light sources 14b and 15b near the proximal end are configured by many luminous bodies (LEDs for example) (similar to the form illustrated in FIG. 2).

Alternatively, the numbers of the respective luminous bodies of the light sources 14a and 15a near the distal end and the light sources 14b and 15b near the proximal end are set to be the same, and control of turning the supply current to the light sources 14b and 15b near the proximal end to a larger current compared to the supply current to the light sources 14a and 15a near the distal end is performed, thereby emitting the large light quantity. Also by the contrivances, the light quantities of the light sources 14a and 15a (first illumination portions) near the distal end and the light sources 14b and 15b (second illumination portions) near the proximal end can be adjusted.

Note that, as another example, setting may be provided by which the light quantity of the first illumination light emitted from the light sources 14a and 15a (first illumination portions) near the distal end can be made smaller than the light quantity of the second illumination light emitted from the light sources 14b and 15b (second illumination portions).

Note that the plurality of light sources (13a, 14a, 14b, 15a, 15b) are electrically connected with the light source portion 21 (to be described later; see FIG. 1) of the processor 20 by a signal line 27. Therefore, the signal line 27 is inserted and arranged from the individual light sources (13a, 14a, 14b, 15a, 15b) at the distal end portion 10a of the endoscope 10 to the insertion portion, the operation portion and the universal cable (not shown in the figure), and is further extended through a connector portion (not shown in the figure; to be described later) to the light source portion 21 inside the processor 20. Thus, for the light sources (13a, 14a, 14b, 15a, 15b), drive control and light quantity adjustment control or the like are performed by the light source portion 21.

The individual image pickup portions including the plurality of image pickup devices (11A, 11B, 11C) receive reflected light from the observation target illuminated by the individual light sources (13a, 14a, 14b, 15a, 15b). The reflected light from the target is transmitted through the image pickup optical system so that an optical image of the observation target is formed, and the optical image is configured to be image-formed on the light receiving surfaces of the individual image pickup devices (11A, 11B, 11C). Then, the image pickup devices (11A, 11B, 11C) perform predetermined photoelectric conversion processing to generate image signals and output the image signals to an image processing portion 24 of the processor 20.

Note that, in the inside of the distal end portion 10a, other than the above-described components, various kinds of components such as a channel opening connected to a treatment instrument channel (not shown in the figure) to insert the insertion portion and a fluid duct for air/water feeding are disposed; however, since the components not described above are parts not directly related to the present invention, the illustration and the detailed descriptions are omitted.

The processor 20 is a configuration unit which functions as a control portion that controls the individual configuration units configuring the present endoscope system 1 and generally controls the entire system, and also functions as a signal processing portion that processes the image signals acquired by the image pickup portions (the plurality of image pickup devices 11A, 11B, 11C) of the endoscope 10, instruction signals from various kinds of operation members and various kinds of control signals or the like.

The processor 20, for example, outputs the control signals for driving the image pickup portions including the image pickup devices 11A, 11B, 11C inside the distal end portion 10a or the like, drives the plurality of light sources (13a, 14a, 14b, 15a, 15b) by the light source portion 21, outputs the control signals for light quantity adjustment or the like, or receives the instruction signals from the various kinds of operation members (not shown in the figure) of the operation portion, and outputs the control signals respectively corresponding to the instruction signals. Further, the processor 20 receives signals outputted from the image pickup portions (image pickup devices 11A, 11B, 11C) for example, performs predetermined signal processing, generates the image signals for display, and generates image data for recording or the like.

Therefore, in the inside of the processor 20, a plurality of electronic circuit board units are provided configuring an image processing portion 24 which is an image signal generation portion configured to receive output signals from the image pickup portions through a signal line 16, perform predetermined image signal processing to the output signals and generate the image signals indicating an endoscope image including the front visual field image (first image) and the side visual field image (second image), an image output portion 25 configured to generate and output the image signals (image data) for display or the like for displaying an image at the display portion 30 on the basis of the image signals generated by the image processing portion 24, the light source portion 21 configured to perform the light quantity adjustment control or the like of the illumination light emitted by driving the plurality of light sources (13a, 14a, 14b, 15a, 15b), and a control portion 23 mounted with a control circuit or the like configured to receive the instruction signals from the other configuration units (not shown in the figure) or the like and execute various kinds of control or the like.

Note that the processor 20 is provided with the connector portion (not shown in the figure), and (the distal end of the universal cable of) the endoscope 10 is freely attached and detached. Then, the connector portion is, though not shown in the figure, similar to that of the endoscope of a conventional and general regular form at a point of being configured including a fluid duct connection portion or the like other than a signal line connection portion which transmits various kinds of electric signals.

Further, in the inside of the processor 20, the power supply portion 26 which is the electric power supply portion configured to receive power supply from an external alternating current (AC) power source and supply the power of a required voltage to the individual configuration units of the present endoscope system 1 is provided. Note that the power supply portion 26 is provided outside the insertion portion.

Then, to the processor 20, the display portion 30 configured to display the endoscope image on the basis of the image signals (image data) outputted from the image output portion 25 and display a display screen or the like when various kinds of setting are to be performed by displaying various kinds of setting items or the like inside the processor 20 is electrically connected.

The display portion 30 is a display device configured to receive the image signals for display from the image output portion 25, display the side visual field image (second image) lined up with the front visual field image (first image), and display the endoscope image including the front visual field image (first image) and the side visual field image (second image) within the same screen.

As the display portion 30, other than a liquid crystal display (LCD) device and an organic electro-luminescence (organic EL:®EL) display device for example, a general display device using a CRT (cathode ray tube (Braun tube)) or the like is applied.

Here, one example of the endoscope image displayed on the display screen of the display portion 30 using the endoscope system 1 of the present embodiment will be simply described using FIG. 3. A display example illustrated in FIG. 3 illustrates a form of displaying the plurality of endoscope images acquired respectively by the image pickup devices (11A, 11B, 11C) respectively included in the plurality of image pickup portions in respectively different display frames.

Here, for example, for the endoscope image displayed on a display screen 30a arranged at a center part in FIG. 3, the front visual field image acquired by the image pickup device 11A for acquiring the front visual field image is displayed. In addition, for the endoscope image displayed on a display screen 30b arranged at one side part of the display screen 30a, one side visual field image acquired by one image pickup device 11B for acquiring the side visual field image is displayed. Then, for the endoscope image displayed on a display screen 30c arranged at the other side part of the display screen 30a, the other side visual field image acquired by the other image pickup device 11C for acquiring the side visual field image is displayed.

Note that the plurality of (three) display screens 30a, 30b and 30c may be in a form of providing and displaying a plurality of (three) display frames within one screen in the single display device, or, differently from the form, may be in a form that the plurality of (three) display devices are lined up and arranged as the display portion 30 and the endoscope images acquired by the individual image pickup portions (image pickup devices) are respectively and separately displayed at the respective display devices. In addition, a form different from the display forms may be the form of combining and displaying the plurality of visual field images that are the front visual field image and the side visual field image as one image on a single screen.

Further, in the case of the configuration including the plurality of display devices (display portions), the following display form is also conceivable. That is, the configuration may be such that the endoscope image can be displayed in the two or more different display forms (display modes), and either one of the display modes is selected and switched to perform display. In this case, as a form example of the display mode, various display forms are conceivable such as a first display mode that one of the plurality of display portions receives the image signals for display from the image output portion 25 and displays the front visual field image (first image) and the side visual field image (second image) within the same screen, and a second display mode that the adjacent different display portions among the plurality of display portions respectively receive the image signals for display from the image output portion 25 and display the front visual field image (first image) and the side visual field image (second image) by the respective different adjacent display portions.

An outline configuration of the endoscope system 1 including the endoscope 10 of the present embodiment is as described above. The configuration omitted in the above descriptions is assumed to have the configuration almost similar to that of the endoscope system which is conventionally and generally in practical use.

In the endoscope 10 of the present embodiment configured in this way, respective irradiation ranges by the plurality of light sources (13a, 14a, 14b, 15a, 15b) are illustrated in FIG. 2. For example, the irradiation range by the plurality of (two) light sources 13a for illuminating the front visual field is an area indicated by a sign [A] in FIG. 2. Then, a display range corresponding to the area is an area indicated by the sign [A] in the display screen 30a in FIG. 3. At the time, areas irradiated by only one of the plurality of (two) light sources 13a, that is, both side areas (a partial area of a sign [AB] and a partial area of [AC]) of the display screen 30a, are irradiated with the illumination light from the light sources 14a and 15a for illuminating the side visual field respectively. By the illumination light from the light sources 14a and 15a, a part each of the front visual field and a rear visual field is illuminated.

The irradiation range by the plurality of (two) light sources 14a and 14b for illuminating the side visual field is an area indicated by a sign [B] in FIG. 2. A display range corresponding to the area is an area indicated by the sign [B] in the display screen 30b in FIG. 3. At the time, a left side area (partial area of the sign [AB]) of the display screen 30b in the area irradiated only by the light source 14a is irradiated with a part of the illumination light from the light source 13a for illuminating the front visual field as described above, and the light source 14b near the proximal end is configured to be able to emit the illumination light of the light quantity different from the light quantity of the light source 14a near the distal end. From the fact, a right side area (area of a sign [B1]) of the display screen 30b in the area irradiated by the light source 14b is irradiated with the illumination light of the light quantity larger than the light quantity of the illumination light emitted to the left side area (partial area of the sign [AB]).

Similarly, the irradiation range by the plurality of (two) light sources 15a and 15b for illuminating the side visual field is an area indicated by a sign [C] in FIG. 2. A display range corresponding to the area is an area indicated by the sign [C] in the display screen 30c in FIG. 3. At the time, a right side area (partial area of a sign [AC]) of the display screen 30c in the area irradiated only by the light source 15a is irradiated with a part of the illumination light from the light source 13a for illuminating the front visual field as described above.

On the other hand, the light source 15b near the proximal end is configured to be able to emit the illumination light of the light quantity larger than the light quantity of the light source 15a near the distal end. From the fact, a left side area (area of a sign [C1]) of the display screen 30c in the area irradiated by the light source 15b is irradiated with the illumination light of the light quantity larger than the light quantity of the illumination light emitted to the right side area (partial area of the sign [AC]).

As described above, according to the first embodiment, in the wide angle endoscope having the front visual field and the side visual field, since the configuration is such that the light quantity of the light sources 14b and 15b near the proximal end among the plurality of light sources (14a, 14b, 15a, 15b) for illuminating the side visual field and the light quantity of the light sources 14a and 15a near the distal end can be set to be different, the endoscope and the endoscope system capable of making a bright part and a dark part in the entire visual field more inconspicuous resulting from imbalance of brightness generated among the plurality of images acquired by the plurality of image pickup portions and capable of acquiring excellent endoscope images at all times can be easily realized by a simple configuration.

Note that, in the first embodiment, an example configured including three image pickup devices and including two each of the light sources for each image pickup device is illustrated, but it is not limited to the form.

### Second Embodiment

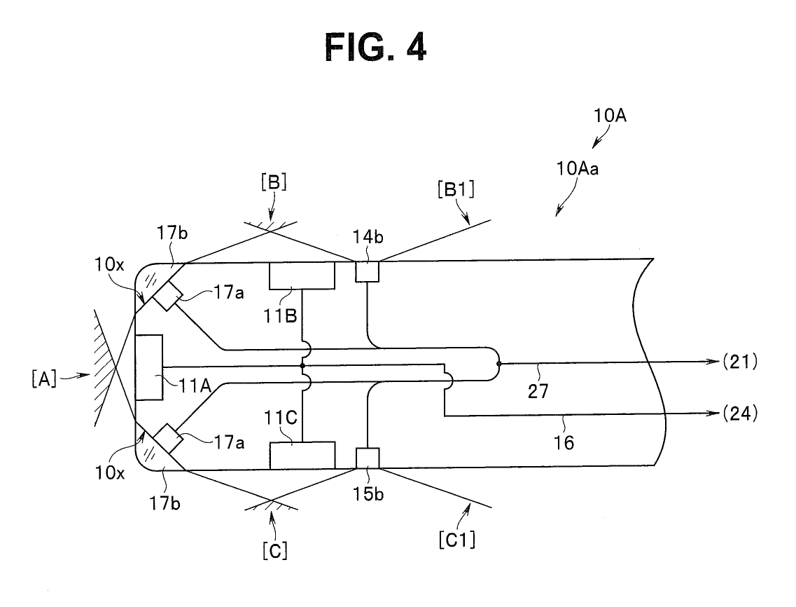

Next, the configuration of the endoscope of the second embodiment of the present invention will be described below. FIG. 4 is a block configuration diagram schematically illustrating a part (the inside of the distal end portion) of the configuration of the endoscope of the second embodiment of the present invention. FIG. 4 illustrates one example of the irradiation range of the illumination light emitted from the individual illumination portions together, similarly to FIG. 2 described above.

The configuration of the endoscope and the endoscope system of the second embodiment is basically formed of the configuration almost similar to that of the first embodiment described above, and only the configuration of the distal end portion in the endoscope and the arrangement of the light sources in the inside are different. Hereinafter, only the configuration different from the first embodiment described above will be described in detail.

In an endoscope 10A of the present embodiment, the point that the plurality of (three) image pickup devices are provided inside a distal end portion 10Aa and are configured as an image pickup device 11A for acquiring the front visual field and image pickup devices 11B and 11C for acquiring the side visual field is similar to that of the first embodiment described above.

In addition, in the endoscope 10A of the present embodiment, outermost edge portions of a most distal end part of the distal end portion 10Aa are formed so as to have inclined planes 10x of an angle of about 45 degrees toward the proximal end side to a distal end face of the distal end portion 10Aa, that is, a plane roughly orthogonal to the insertion axis direction.

The inclined planes 10x are formed respectively at parts facing each other across the image pickup device 11A for acquiring the front visual field. To each of the two inclined planes 10x, an optical member 17b which is a wide angle illumination optical system formed to have a wide light distribution characteristic for example is disposed.

Note that specific examples of the optical member 17b having the wide light distribution characteristic are a fish-eye lens, a light diffusion lens or the like. Among them, as a specific configuration example as the light diffusion lens, for example, various kinds of means can be applied such as arranging a plurality of members in a shape of glass beads on a surface of a transparent resin member, working a surface so as to form a rugged fine pattern, using a Fresnel lens, and sticking a light diffusion sheet.

Then, on an inner side of the optical member 17b provided on each of the two inclined planes 10x, a light source 17a is arranged respectively. In this case, the respective light sources 17a are light sources that irradiate the predetermined range (at least one of the front and the side) of the front and the side in the insertion axis direction of the endoscope insertion portion with the illumination light, and similarly to the first embodiment described above, the luminous body such as the light emitting diode (LED) is applied. By the configuration, the respective light sources 17a illuminate an area [A] of the front visual field and an area [B] of the side visual field illustrated in FIG. 4.

That is, the distal end portion 10Aa of the insertion portion of the endoscope 10A is configured including a front surface including a surface crossing the longitudinal direction of the insertion portion and a side surface including a surface roughly parallel to the longitudinal axis direction of the insertion portion. Then, the light source 17a (that is, the first illumination portion) is arranged at a part where the insertion portion front surface and the insertion portion side surface cross. Then, the light source 17a (first illumination portion) includes the optical member 17b (wide angle illumination optical system) that simultaneously irradiates the front (first direction) of the insertion portion and the side (second direction) of the insertion portion.

On the other hand, at respective parts near the proximal end, which are vicinities of the respective image pickup devices 11B and 11C for acquiring the side visual field image, the light sources 14b and 15b for illuminating the side visual field are disposed. The light sources 14b and 15b are the light sources which irradiate the predetermined range of the side in the radial direction of the insertion portion that is the direction roughly orthogonal to the insertion axis direction of the endoscope insertion portion with the illumination light. Note that, in the present embodiment, the light quantities of the individual light sources 14b and 15b to the light quantities of the individual light sources 17a are set to be almost equal. The other configuration is completely similar to that of the first embodiment described above.

In the endoscope 10A of the present embodiment configured in this way, the respective irradiation ranges by the plurality of light sources (17a, 14b, 15b) are as illustrated in FIG. 4. For example, by the plurality of (two) light sources 17a, the irradiation range (area indicated by a sign [A] in FIG. 4) in the front visual field is illuminated. In addition, by one of the light sources 17a and the light source 14b, one irradiation range (area indicated by a sign [B] in FIG. 4) of the side visual field is illuminated. Similarly, by the other one of the light sources 17a and the light source 15b, the other irradiation range (area indicated by a sign [C] in FIG. 4) of the side visual field is illuminated. By such a configuration, the light quantity of the illumination light at parts (the respective areas corresponding to the signs [AB] and [AC] illustrated in FIG. 2) where the display image of the front visual field image and the display image of the side visual field image are superimposed is set to be the light quantity almost equal to the light quantity of the side visual field illumination light in the areas (the respective areas indicated by signs [B1] and [C1] in FIG. 4) near the proximal end in the display image of the side visual field image. Therefore, thus, also by the present embodiment, effects similar to the effects of the first embodiment described above can be obtained.

### Third Embodiment

Next, the configuration of the endoscope and the endoscope system of the third embodiment of the present invention will be described below. In the first and second embodiments described above, the luminous body such as the light emitting diode (LED) is applied as the light source of the illumination light and the configuration example in which the luminous body is disposed inside the distal end portion of the insertion portion of the endoscope is presented. In contrast, in the third embodiment of the present invention described below, a system form configured to apply a luminous body such as a xenon lamp which is conventionally and generally applied as the light source of the illumination light, provide the light source in a processor or the like and guide the illumination light from the light source to the distal end portion of the insertion portion of the endoscope using a light guide is illustrated.

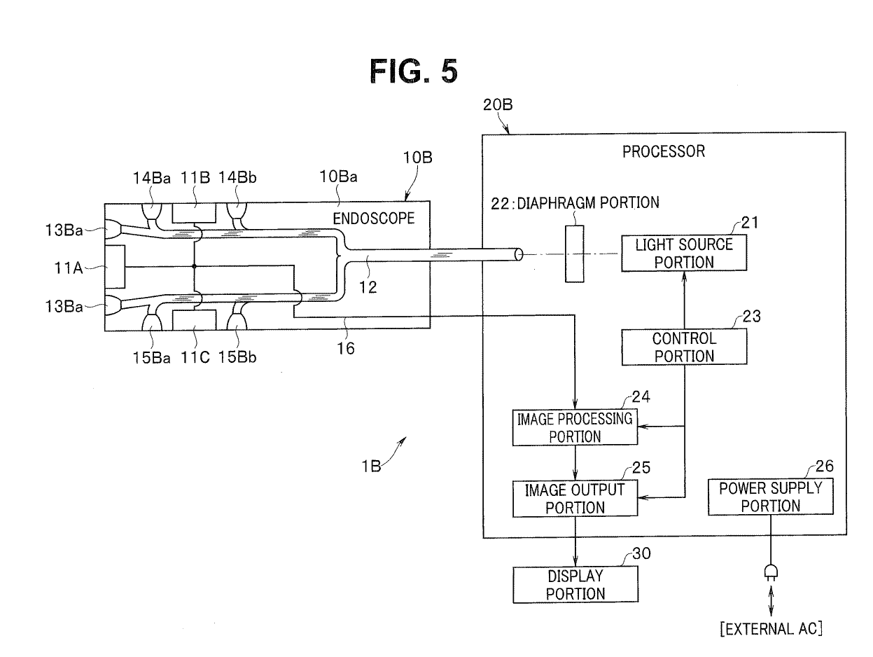

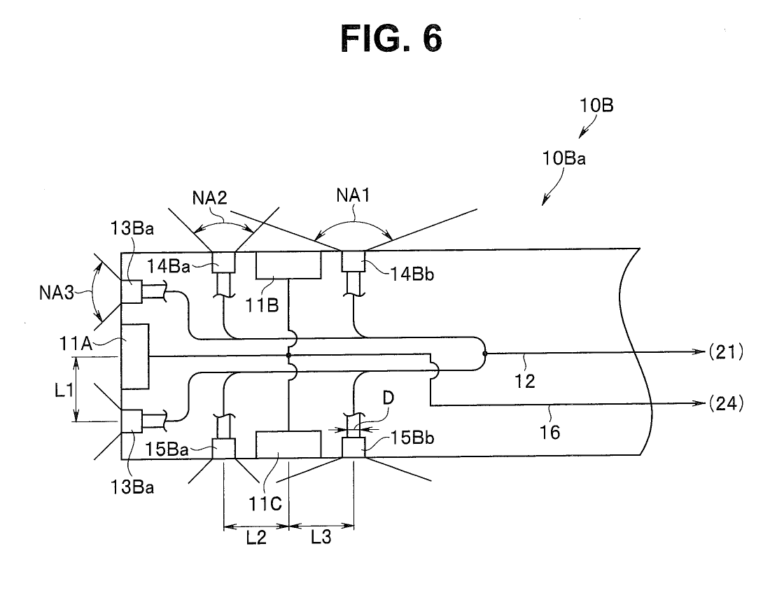

FIG. 5 is a block configuration diagram schematically illustrating the entire configuration of the endoscope system of the third embodiment of the present invention. FIG. 6 is a block configuration diagram schematically illustrating the internal configuration of the distal end portion of the insertion portion of the endoscope in the endoscope system of the present embodiment, and illustrates one example of the irradiation range of the illumination light emitted from the individual illumination portions together.

The basic configuration of the endoscope system of the present embodiment is almost similar to that of the first and second embodiments described above, and as described above, the point of the configuration using the different light source is different. Hereinafter, only the configuration different from the first and second embodiments described above will be described in detail.

As illustrated in FIG. 5, the endoscope system 1B of the present embodiment is mainly configured by an endoscope 10B of the present embodiment, a processor 20B and the display portion 30 or the like. Note that, since the other configuration units in the endoscope system 1B of the present embodiment are parts not directly related to the present invention, the illustration and the detailed descriptions are omitted.

For the endoscope 10B, the endoscope of the conventional and general form configured by the insertion portion, the operation portion and the universal cable or the like is applied. That is, the insertion portion is formed of the elongated tubular configuration unit formed by connecting a distal end portion 10Ba, the bending portion, and the flexible tube portion in order from the distal end side. The proximal end of the insertion portion is connected to the distal end of the operation portion. The insertion portion is the configuration portion to be inserted to the inside of a lumen (inside of a body cavity) of the subject when the endoscope 10B is used. The distal end portion 10Ba is disposed on the most distal end side of the insertion portion, is configured by the rigid member, and is the configuration unit with various kinds of configuration members arranged on the outer surface and in the inside.

The endoscope 10B in the endoscope system 1B of the present embodiment is configured to observe and pick up the plurality of images of the different visual fields which are the front visual field and the side visual field. Therefore, in the inside of the distal end portion 10Ba of the insertion portion of the endoscope 10B, the image pickup portions configured including the plurality of image pickup devices (11A, 11B, 11C) and the image pickup optical system or the like, a light guide cable 12 configured to supply the illumination light emitted from the light source portion 21 to be described later to a plurality of illumination windows (13Ba, 14Ba, 14Bb, 15Ba, 15Bb) to be described later and guide the light, and the plurality of illumination windows (13Ba, 14Ba, 14Bb, 15Ba, 15Bb) for emitting the illumination light emitted from a distal end of the light guide cable 12 toward the outside or the like are disposed.

Here, the arrangement configuration of the plurality of image pickup devices (11A, 11B, 11C), the light guide cable 12, and the plurality of illumination windows (13Ba, 14Ba, 14Bb, 15Ba, 15Bb) or the like in the inside of the distal end portion 10Ba of the present endoscope 10B will be simply described below mainly using FIG. 6.

The image pickup device indicated by the sign 11A among the plurality of image pickup devices (11A, 11B, 11C) is the image pickup device for acquiring the front visual field image, the light receiving surface of which is arranged toward the front, in order to acquire the front visual field image with the front in the insertion axis direction of the endoscope insertion portion as the observation visual field. In addition, the image pickup devices indicated by the signs 11B and 11C are the image pickup devices for acquiring the side visual field image, the light receiving surfaces of which are arranged toward the side respectively, in order to acquire the side visual field image with the side in the radial direction of the insertion portion that is the direction roughly orthogonal to the insertion axis direction of the endoscope insertion portion as the observation visual field. Here, the image pickup device 11B and the image pickup device 11C are arranged oppositely back to back such that the individual light receiving surfaces oppose each other, and are configured to acquire the side visual field image respectively in the directions opposite to each other with the insertion axis direction as the center. That is, the configuration of the image pickup portions in the endoscope of the present embodiment is similar to that of the first and second embodiments described above.

On the other hand, among the plurality of illumination windows (13Ba, 14Ba, 14Bb, 15Ba, 15Bb), the plurality of (two) illumination windows indicated by a sign 13Ba are the illumination windows for illuminating the front visual field, which irradiate the predetermined range of the front in the insertion axis direction of the endoscope insertion portion with the illumination light. The plurality of (two) illumination windows 13Ba are arranged at both side parts of the image pickup device 11A for acquiring the front visual field image so as to hold the image pickup device 11A in between, within the plane roughly orthogonal to the insertion axis direction.

In addition, the plurality of (two) illumination windows indicated by signs 14Ba and 14Bb are the illumination windows for illuminating the side visual field, which irradiate the predetermined range of one side (the right side when the distal end side is viewed from the proximal end side regarding the insertion axis direction for example) in the radial direction of the insertion portion that is the direction roughly orthogonal to the insertion axis direction of the endoscope insertion portion with the illumination light. The plurality of (two) illumination windows 14Ba and 14Bb are arranged at both side parts of one image pickup device 11B for acquiring the side visual field image so as to hold the image pickup device 11B in between, within the plane parallel with the insertion axis direction.

Then, the plurality of (two) illumination windows indicated by signs 15Ba and 15Bb are the illumination windows for illuminating the side visual field, which irradiate the predetermined range of the other side (the left side when the distal end side is viewed from the proximal end side regarding the insertion axis direction for example) in the radial direction of the insertion portion that is the direction roughly orthogonal to the insertion axis direction of the endoscope insertion portion with the illumination light. The plurality of (two) illumination windows 15Ba and 15Bb are arranged at both side parts of the other image pickup device 11C for acquiring the side visual field image so as to hold the image pickup device 11C in between, within the plane parallel with the insertion axis direction.

The distal end side of the light guide cable 12 is structured to be branched in a plurality of directions in the inside of the distal end portion 10Ba of the insertion portion of the endoscope 10B, as illustrated in FIG. 5. The distal end of a part of the branching structure of the light guide cable 12 is arranged in the vicinity of the plurality of (two) illumination windows 13Ba for illuminating the front visual field. In addition, the distal end of another part of the light guide cable 12 is arranged in the vicinity of the plurality of illumination windows 14Ba, 14Bb, 15Ba and 15Bb for illuminating the side visual field.

By such a configuration, the illumination light guided from the light source portion 21 (to be described later) by the light guide cable 12 is branched inside the distal end portion 10Ba of the insertion portion of the endoscope 10B, is guided to the individual illumination windows (13Ba, 14Ba, 14Bb, 15Ba, 15Bb), is then emitted to the individual predetermined directions, and illuminates the predetermined range including the observation target present at a position opposing the individual illumination windows (13Ba, 14Ba, 14Bb, 15Ba, 15Bb).

Then, in the present embodiment, the plurality of illumination windows 13Ba, 14Ba, 14Bb, 15Ba and 15Bb are configured as follows. That is, as illustrated in FIG. 6, in the case of indicating a light distribution angle (irradiation angle) of the illumination windows 14Bb and 15Bb near the proximal end among the plurality of illumination windows 14Ba, 14Bb, 15Ba and 15Bb for illuminating the side visual field by a sign NA1, indicating the light distribution angle (irradiation angle) of the illumination windows 14Ba and 15Ba near the distal end by a sign NA2, and indicating the light distribution angle (irradiation angle) of the two illumination windows 13Ba for illuminating the front visual field by a sign NA3, the setting of the light distribution angles of the individual illumination windows is set to be

NA1>NA2

NA1>NA3.

In this case, in particular, when

[NA2/2]+[NA3/2]≦90°

is set, the illumination light from the illumination windows 14Ba and 15Ba (the sign NA2) near the distal end and the illumination light from the two illumination windows 13Ba (the sign NA3) for illuminating the front visual field are not superimposed.

In addition, the individual image pickup portions including the plurality of image pickup devices (11A, 11B, 11C) receive the reflected light from the observation target illuminated by the individual illumination windows (13Ba, 14Ba, 14Bb, 15Ba, 15Bb), and in response, the image pickup optical system forms the optical image of the observation target on the light receiving surfaces of the individual image pickup devices (11A, 11B, 11C), and the individual image pickup devices (11A, 11B, 11C) perform the photoelectric conversion processing to generate the image signals and output the image signals to the image processing portion 24 of the processor 20B (details will be described later).

Note that, in the inside of the distal end portion 10Ba, other than the structures, various kinds of structures such as the channel opening connected to the treatment instrument channel (not shown in the figure) to insert the insertion portion are disposed; however, since the structures are parts not directly related to the present invention, the illustration and the detailed descriptions are omitted.

The processor 20B is the configuration unit which functions as the control portion that controls the individual configuration units configuring the present endoscope system 1B and generally controls the entire system, and also functions as the signal processing portion that processes the signals acquired by (the plurality of image pickup devices 11A, 11B, 11C of) the image pickup portions of the endoscope 10B, the instruction signals from the various kinds of operation members and the various kinds of control signals or the like.

The processor 20B, for example, outputs the control signals for driving the image pickup portions including the image pickup devices 11A, 11B, 11C inside the distal end portion 10Ba or the like, receives the instruction signals from the various kinds of operation members of the operation portion, and outputs the control signals respectively corresponding to the instruction signals. In addition, the processor 20B receives the image signals outputted from the image pickup portions (image pickup devices 11A, 11B, 11C) for example, performs the predetermined signal processing, generates the image signals for display, and generates the image data for recording or the like.

Therefore, in the inside of the processor 20B, the plurality of electronic circuit board units are provided configuring the image processing portion 24 configured to receive the output signals (image signals) from the image pickup portions through a signal line 16, perform the predetermined image signal processing to the output signals and generate the image signals indicating the endoscope image including the front visual field image (first image) and the side visual field image (second image), the image output portion 25 configured to generate and output the image signals (image data) for display or the like for displaying the image at the display portion 30 on the basis of the image signals generated by the image processing portion 24, and the control portion 23 mounted with the control circuit or the like configured to receive the instruction signals from the other configuration units or the like and execute various kinds of control or the like. In addition to the portions, in the inside of the processor 20B, various kinds of configuration units such as the light source portion 21 and a diaphragm portion 22 are disposed further.

Among the configuration units, the light source portion 21 is the configuration unit including the light source formed of the luminous body which emits the illumination light. The light source portion 21 is the configuration unit including a main light source formed of the luminous body such as a xenon lamp, a halogen lamp and the light emitting diode (LED), and including the control circuit or the like which performs the light quantity adjustment control of the illumination light emitted from the main light source or the like, similarly to the one equipped in the endoscope system of the conventional and general configuration.

The diaphragm portion 22 is the configuration unit which adjusts the light quantity of the illumination light emitted from the light source of the light source portion 21 under the control of the control portion 23.

Then, to the light source portion 21, the light guide cable 12 is optically connected. The light guide cable 12 is a light guide body which guides the illumination light emitted from the light source of the light source portion 21 and made to pass through the diaphragm portion 22 to the distal end portion 10Ba of the insertion portion of the endoscope 10B, and is formed in a cable shape by binding fibrous light guides for examples. Then, as described above, the light guide cable 12 is branched toward the plurality of individual illumination windows inside the distal end portion 10Ba.

Note that the processor 20B is provided with the connector portion (not shown in the figure), and (the universal cable of) the endoscope 10B is freely attached and detached. Then, the connector portion is, though not shown in the figure, configured including the fluid duct connection portion and a light guide connection portion for illumination light supply or the like other than the signal line connection portion which transmits various kinds of electric signals. The specific configuration is similar to that of the endoscope of the conventional and general regular form.

In addition, in the inside of the processor 20B, the power supply portion 26 configured to receive the power supply from the external alternating current (AC) power source and supply the power of the required voltage to the individual configuration units of the present endoscope system 1B is provided.

Then, to the processor 20B, the display portion 30 configured to display the endoscope image on the basis of the image signals (image data) outputted from the image output portion 25 and display the display screen or the like when various kinds of setting are to be performed by displaying various kinds of setting items or the like inside the processor 20B is electrically connected. As the display portion 30, other than the liquid crystal display (LCD) device and the organic electro-luminescence (organic EL: OEL) display device for example, the general display device using the CRT (cathode ray tube (Braun tube)) or the like is applied.

Also in the endoscope system 1B of the present embodiment configured in this way, the endoscope image displayed on the display screen of the display portion 30 is in a form similar to the form described in the first embodiment or the like described above (see the display example in FIG. 2).

The outline configuration of the endoscope system 1B including the endoscope 10B of the present embodiment is as described above. The configuration omitted in the above descriptions is assumed to have the configuration almost similar to that of the endoscope system of the first and second embodiments or the endoscope system which is conventionally and generally in practical use.

As described above, according to the third embodiment, the effects similar to the effects of the first and second embodiments described above can be obtained. In addition, in the present embodiment, for the plurality of illumination windows 13Ba, 14Ba, 14Bb, 15Ba and 15Bb, in the case of indicating the light distribution angle (irradiation angle) of the illumination windows 14Bb and 15Bb near the proximal end among the plurality of illumination windows 14Ba, 14Bb, 15Ba and 15Bb for illuminating the side visual field by the sign NA1, indicating the light distribution angle (irradiation angle) of the illumination windows 14Ba and 15Ba near the distal end by the sign NA2, and indicating the light distribution angle (irradiation angle) of the two illumination windows 13Ba for illuminating the front visual field by the sign NA3, the setting of the light distribution angles of the individual illumination windows is set to be NA1>NA2, NA1>NA3, and [NA2/2]+[NA3/2]≦90°.

By the configuration, since the light distribution angle NA2 of the illumination light emitted from the illumination windows 14Ba and 15Ba near the distal end and the light distribution angle NA3 of the illumination light emitted from the two illumination windows 13Ba for illuminating the front visual field are set to be narrow in contrast with the light distribution angle NA1 of the illumination light emitted from the illumination windows 14Bb and 15Bb near the proximal end among the plurality of illumination windows 14Ba, 14Bb, 15Ba and 15Bb for illuminating the side visual field, the illumination light does not overlap with each other at a boundary part of each illumination light. Therefore, on the displayed endoscope image, illumination unevenness is not generated in boundary areas (see the signs [AB] and [AC] in FIG. 3) of each illumination light. In addition, on the endoscope image, even in both outer edge areas (see the signs [B1] and [C1] in FIG. 3), the brightness can be sufficiently secured compared to an almost center area, and thus the dark part in contrast with the bright part in the entire image can be made more inconspicuous.

Note that, in the case of the configuration, the illumination light emitted from the individual illumination windows sometimes does not sufficiently overlap. However, it is sufficient to set the irradiation angle of the illumination light so as to cover an image pickup range of the image pickup devices, and even when an irradiation light quantity becomes insufficient outside the image pickup range of the image pickup devices, the obtained endoscope image is not affected at all and there is no problem.

Now, the third embodiment is configured to set the light distribution angles of the individual illumination windows to the predetermined setting, but is not limited to the configuration. For example, in the basic configuration that the xenon lamp or the like is the light source, the light source is provided inside the processor 20B as the light source portion 21, and the illumination light of the light source is guided to the distal end portion using the light guide cable 12 similarly to the third embodiment, the configuration may be such that the light quantity of the illumination light emitted from the parts near the proximal end of the image pickup devices 11B and 11C for acquiring the side visual field image, that is, the illumination light emitted from the illumination windows 14Bb and 15Bb near the proximal end in the present embodiment, and the light quantity of the illumination light emitted from the illumination windows 14Ba and 15Ba near the distal end are set to be different, similarly to the first and second embodiments described above.

In this case, as the contrivance of adjusting the light quantity of the illumination light emitted from the individual illumination windows 14Ba, 14Bb, 15Ba and 15Bb, for example, at the part after branching of the light guide cable 12 which guides the illumination light from the light source portion 21, the following configuration is conceivable. That is, a diameter of the light guide cable 12 is defined as D, and a cross-sectional area of the light guide cable 12 is defined as S. In this case, the diameter D of the light guide cable 12 is determined by the number of strands such as light guide fibers to be bundled. In addition, the cross-sectional area S of the light guide cable 12 is determined by the diameter D. Therefore, in the case of setting the light quantity of the illumination light emitted from the illumination windows 14Bb and 15Bb near the proximal end to be larger than the light quantity of the illumination light emitted from the illumination windows 14Ba and 15Ba near the distal end for example, when the diameter is D1 and the cross-sectional area is S1 for the light guide cable 12 arranged in the vicinity of the illumination windows 14Bb and 15Bb near the proximal end, the diameter is D2 and the cross-sectional area is S2 for the light guide cable 12 arranged in the vicinity of the illumination windows 14Ba and 15Ba near the distal end, and the diameter is D3 and the cross-sectional area is S3 for the light guide cable 12 arranged in the vicinity of the two illumination windows 13Ba for illuminating the front visual field, the setting is performed to be

D1>D2

D1>D3

or

S1>S2

S1>S3.

In addition, as another contrivance of the light quantity adjustment, following means is also conceivable. Generally, the light quantity of the illumination light tends to decrease as reaching a peripheral area separated from the center part with the almost center part as a peak of the brightness within the irradiation range. Therefore, by adjusting the arrangement of the light source to the image pickup device, that is, a distance between the image pickup device and the light source, the light quantity of the illumination light which irradiates an image range acquired by the image pickup device can be adjusted.

Then, for the arrangement of the plurality of illumination windows 13Ba, 14Ba, 14Bb, 15Ba and 15Bb, the following configuration is conceivable. That is, in the case of indicating a distance between the image pickup device 11A for acquiring the front visual field image and the two illumination windows 13Ba for illuminating the front visual field by L1, indicating the distance between the image pickup devices 11B and 11C for acquiring the side visual field image and the illumination windows 14Ba and 15Ba near the distal end among the plurality of illumination windows 14Ba, 14Bb, 15Ba and 15Bb for illuminating the side visual field by L2, and indicating the distance between the image pickup devices 11B and 11C for acquiring the side visual field image and the illumination windows 14Bb and 15Bb near the proximal end by L3, the setting is performed to be

L1>L3

L2>L3.

Also in the case of the respective configurations described above, since the light quantity of the illumination light emitted from the illumination windows 14Bb and 15Bb near the proximal end can be made larger than the light quantity of the illumination light emitted from the illumination windows 14Ba and 15Ba near the distal end, similar to the third embodiment, the dark part in contrast with the bright part in the entire image can be made more inconspicuous.

Note that the present invention is not limited to the embodiments described above and of course various modifications and applications can be executed without departing from the gist of the invention. Further, the embodiments include the inventions in various stages, and various inventions can be extracted by appropriate combinations in a plurality of disclosed constituent elements. For example, even when some constituent elements are deleted from the entire constituent elements indicated in the one embodiment, in the case that the problem to be solved by the invention can be solved and the effect of the invention can be obtained, the configuration from which the constituent elements are deleted can be extracted as the invention. Further, components over the different embodiments may be appropriately combined. The invention is not constrained by the specific embodiment other than being limited by attached claims.

### INDUSTRIAL APPLICABILITY

The present invention is applicable not only to an endoscope control device in a medical field but also an endoscope control device in an industrial field.

What is claimed is:

1. An endoscope comprising:
an insertion portion configured to be inserted to an inside of a subject;
a first observation portion provided on a front surface of the insertion portion and configured to observe a first area including a front which is a longitudinal axis direction of the insertion portion in an observation target;
a second observation portion provided on a side surface of the insertion portion and configured to observe a second area including a side which is a direction crossing the longitudinal axis direction of the insertion portion in the observation target and be adjacent to the first area;
a first illumination portion arranged on a distal end side with respect to the second observation portion on the side surface of the insertion portion and configured to irradiate the second area of the observation target with first illumination light; and
a second illumination portion arranged on a proximal end side with respect to the second observation portion on the side surface of the insertion portion and configured to irradiate the second area of the observation target with second illumination light,
wherein the second illumination portion performs irradiation with the second illumination light of a light quantity almost equal to a light quantity for which the first illumination light in a range observed in the second observation portion and illumination light from an illumination portion provided on the front surface of the insertion portion and configured to irradiate at least the first area with the illumination light are put together.

2. The endoscope according to claim 1, further comprising:
a light source portion provided outside the insertion portion and configured to emit light; and
a light guide configured to supply the light to the first illumination portion and the second illumination portion.

3. The endoscope according to claim 1, further comprising:
a power supply portion provided outside the insertion portion and configured to supply power;
at least one first light emitting elements provided on the first illumination portion and configured to perform irradiation with the first illumination light based on the power supplied from the power supply portion; and
at least one second light emitting element provided on the second illumination portion and configured to perform irradiation with the second illumination light based on the power supplied from the power supply portion.

4. The endoscope according to claim 1,
wherein the first illumination portion includes a wide angle illumination optical system configured to simultaneously irradiate the first area and the second area.

5. The endoscope according to claim 1,
wherein the second observation portion and the second illumination portion are arranged respectively on a plane set to be parallel with a longitudinal axis of the insertion portion.

6. The endoscope according to claim 1,
wherein, in a case that a light distribution angle of the second illumination portion is NA1 and a light distribution angle of the first illumination portion is NA2,
NA1>NA2
is set.

7. The endoscope according to claim 1,
wherein, in a case that a distance between the second observation portion and the first illumination portion is L2 and
a distance between the second observation portion and the second illumination portion is L3,
L2>L3
is set.

8. An endoscope system comprising:
the endoscope according to claim 1;
an image signal generation portion configured to generate image signals for which a first image that is an image of the observation target in the first area and a second image that is an image of the observation target in the second area are lined up such that the first image and the second image are arranged at adjacent positions; and
a display portion configured to display the image signals generated by the image signal generation portion.

9. The endoscope system according to claim 8,
wherein the display portion is further provided in plurality, and
display is performed in a display mode which is either one of a first display mode in which one of the plurality of display portions receives the image signals and displays the first image and the second image within a same screen, and a second display mode in which the adjacent different display portions of the plurality of display portions receive the image signals respectively and display the first image and the second image at the different adjacent display portions respectively.

10. The endoscope system according to claim 8,
wherein the first observation portion includes a first image pickup portion configured to photoelectrically convert the first image, and
the second observation portion includes a second image pickup portion configured to photoelectrically convert the second image and be different from the first image pickup portion.
